SEQUENCE LISTING (10 sequences)

SEQ ID NO: 1 - DNA, 280 bp, Homo sapiens
  source 1..280: mol_type = genomic DNA
gggggcgggcggagccttggggtcaccaagagccactgaacgagggtaaggagcggccaccgcgcggactccgcaaccttcccgggatagcttccgtgtccttggctcagctgtgtatgcccgtggtgcgagcactgcggagccgcctggggcgcaagccgggtcttcaccagtccccgggcgcgctgtcggtagggcgggcgctctcgggccaccgggcgggaggtggccgcgtccccagacactggctgggccagagtgagtttagcgcgtaggcggc

SEQ ID NO: 2 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTMQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 3 - protein, 1512 aa, synthetic construct
EELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTMQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 4 - protein, 718 aa, synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 5 - protein, 4 aa, synthetic construct
GGGS

SEQ ID NO: 6 - protein, 11 aa, synthetic construct
GGGSGGGGSRS

SEQ ID NO: 7 - protein, 16 aa, synthetic construct
GGGSGGGGSGGGGSRS

SEQ ID NO: 8 - protein, 21 aa, synthetic construct
GGGSGGGGSGGGGSGGGGSRS

SEQ ID NO: 9 - protein, 26 aa, synthetic construct
GGGSGGGGSGGGGSGGGGSGGGGSRS

SEQ ID NO: 10 - protein, 31 aa, synthetic construct
GGGSGGGGSGGGGSGGGGSGGGGSGGGGSRS

(19) United States
(12) Patent Application Publication
(10) Pub. No.: US 2025/0115884 A1
(43) Pub. Date: Apr. 10, 2025

(54) TAL-Tet1 Fusion Proteins and Methods of Use Thereof

(71) Applicant: The General Hospital Corporation, Boston, MA (US)

(72) Inventors: J. Keith Joung, Winchester, MA (US); Morgan Maeder, Brookline, MA (US); James Angstman, Charlestown, MA (US)

(21) Appl. No.: 18/916,932

(22) Filed: Oct. 16, 2024

Related U.S. Application Data
(63) Continuation of application No. 17/149,458, filed on Jan. 14, 2021, now Pat. No. 12,152,254.
Application No. 17/149,458 is a continuation of application No. 15/894,004, filed on Feb. 12, 2018, now Pat. No. 10,894,950.
Application No. 15/894,004 is a division of application No. 13/838,520, filed on Mar. 15, 2013, now Pat. No. 9,890,364.
(60) Provisional application No. 61/652,711, filed on May 29, 2012.

(51) Int. Cl. C12N 9/02

(52) U.S. Cl. CPC C12N 9/0071; C07K 2319/80; C07K 2319/81

(57) ABSTRACT

Fusion proteins comprising a DNA binding domain, e.g., a TAL effector repeat array or zinc finger, and a catalytic domain comprising a sequence that catalyzes hydroxylation of methylated cytosines in DNA, and methods of use thereof.

### CLAIM OF PRIORITY

This application is a continuation of U.S. patent application Ser. No. 17/149,458, filed Jan. 14, 2021, which is a continuation of U.S. patent application Ser. No. 15/894,004, filed Feb. 12, 2018, now U.S. Pat. No. 10,894,950, which is a divisional of U.S. patent application Ser. No. 13/838,520, filed Mar. 15, 2013, now U.S. Pat. No. 9,890,364, which claims priority under 35 USC §119(e) to U.S. Patent Application Ser. No. 61/652,711, filed on May 29, 2012, the entire contents of which are hereby incorporated by reference.

### FEDERALLY SPONSORED RESEARCH OR DEVELOPMENT

This invention was made with Government support under Grant No. DP1 OD006862 awarded by the National Institutes of Health. The Government has certain rights in the invention.

### SEQUENCE LISTING

This application contains a Sequence Listing that has been submitted electronically as an XML file named “40978-0065004_SL ST26.XML.” The XML file, created on Oct. 15, 2024, is 14,351 bytes in size. The material in the XML file is hereby incorporated by reference in its entirety.

### TECHNICAL FIELD

This invention relates to fusion proteins comprising a DNA binding domain, e.g., a TAL effector repeat array-Tet1 (TAL-Tet1) or zinc finger, and a catalytic domain comprising a sequence that catalyzes hydroxylation of methylated cytosines in DNA, and methods of use thereof.

BACKGROUND

Tet1 is an enzyme that catalyzes the conversion of 5-methylcytosine (5mC) to 5-hydroxymethylcytosine (5hmC) (Tahiliani, M. et al. Science 324, 930-935 (2009)). While the role of 5hmC is not entirely clear, it has been proposed that it may be an intermediate in the process of demethylation of 5-methylcytosine to cytosine. This is supported by evidence that overexpression of Tet1 in cultured cells leads to an overall decrease in levels of 5mC (Tahiliani, M. et al. Science 324, 930-935 (2009)). Several mechanisms of demethylation have been proposed. There is evidence that 5hmC can be deaminated and that the resulting mismatched base is recognized by DNA glycosylases and subsequently repaired to cytosine via the base excision repair pathway (Guo et el., Cell 145, 423-434 (2011)). Alternatively, there is also evidence that iterative oxidation of 5hmC by Tet1 yields 5-formylcytosine (fC) and 5-carboxylcytosine (caC), which can then be recognized by thymine DNA glycosylase and reverted to cytosine through base excision repair (He et al., Science 333, 1303-1307 (2011)). In either case, all evidence highlights Tet1 as a primary catalyst for DNA demethylation.

### SUMMARY

At least in part, the present invention is based on the discovery of fusion proteins comprised of an engineered custom DNA-binding domain fused to a protein capable of catalyzing hydroxylation of methylated cytosines in DNA in vitro, in cells, or in whole organisms. Such a hybrid protein would lead to formation of hydroxymethylated cytosines from methylated cytosines in DNA and subsequent demethylation of the cytosines in living cells or organisms.

Thus provided herein are fusion proteins comprising an engineered DNA-binding domain that binds specifically to a preselected target sequence, and a catalytic domain comprising a sequence that catalyzes demethylation of cytosines in DNA. In some embodiments, the fusion proteins further comprise a linker between the DNA binding domain and the catalytic domain.

In some embodiments, the DNA-binding domain is or comprises an engineered transcription activator-like (TAL) effector repeat array or a zinc finger.

In some embodiments, the catalytic domain comprises a catalytic domain of Tet1, Tet2, or Tet3. In some embodiments, the catalytic domain comprises amino acids 1580-2052 of Tet1, amino acids 1290-1905 of Tet2, amino acids 966-1678 of Tet3, amino acids 1418-2136 of Tet1, amino acids 1-2136 of Tet1, or amino acids 1-584 and 625-2136 of Tet1.

In some embodiments, the fusion proteins comprise a plurality of catalytic domains.

In a further aspect, the invention provides methods for reducing methylation of a selected DNA sequence in a mammalian cell, the method comprising contacting the cell with a fusion protein comprising an engineered DNA-binding domain that binds specifically to a target sequence, wherein the target sequence is within about 10 kb, 5 kb, 2 kb, or 1 kb, 500 bp, 250 bp, 100 bp, 50 bp, 40 bp, 30 bp, or 20 bp, of the selected DNA sequence, and a catalytic domain comprising a sequence that catalyzes hydroxylation of methylated cytosines in DNA.

In another aspect, the invention provides methods for reducing methylation of a selected DNA sequence in a mammalian cell, the method comprising contacting the cell with a nucleic acid encoding a fusion protein comprising an engineered DNA-binding domain that binds specifically to a target sequence, wherein the target sequence is within about 10 kb, 5 kb, 2 kb, 1 kb, 500 bp, 250 bp, 100 bp, 50 bp, 40 bp, 30 bp, or 20 bp, of the selected DNA sequence, and a catalytic domain comprising a sequence that catalyzes hydroxylation of methylated cytosines in DNA.

In some embodiments, the fusion protein further comprises a linker between the DNA binding domain and the catalytic domain.

In some embodiments, the DNA-binding domain is or comprises an engineered transcription activator-like (TAL) effector repeat array or a zinc finger.

In some embodiments, the catalytic domain comprises a catalytic domain of Tet1, e.g., amino acids 1418-2136 of Tet1.

In some embodiments, the cell is a human cell.

In some embodiments, the cell is in a living mammal.

In some embodiments, the selected DNA sequence is a sequence of a gene listed in FIG. 7.

In some embodiments, the gene is selected from the group consisting of RASSF1A, MGMT, BRCA1, p16, CDH1, TIMP-3, p15, p73, DAPK, APC, and RELN.

In some embodiments, the cell is present in an individual diagnosed with a disease listed in FIG. 7, and the selected DNA sequence is a gene associated with that disease as listed in FIG. 7.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Structure of Tet1 and TAL-Tet1 fusion proteins.

A) Schematic shows the predicted domain architecture of the Tet1 protein (Tet1-FL), the catalytic domain of the Tet1 protein (Tet1-CD) and the truncated version of Tet1 in which the CXXC domain has been removed (Tet1-NZ). Features shown include the CXXC-type zinc-binding domain (CXXC), three nuclear localization signals (NLS), the cysteine-rich region (cys-rich) and the double-stranded β helix domain (DSBH) (Tahiliani et al., Science 2009). B) Schematic shows an exemplary TAL-Tet1 fusion protein, in which Tet1 is fused to the C-terminal end of the TALE protein via a Gly₃Ser linker. In some embodiments, the Tet1 is fused to the N-terminal end. The engineered TALE protein binds DNA target sequence and thus localizes Tet1 protein to this region.

FIG. 2. TAL Arrays Targeted to Human Klf4. Sequence of human Klf4 intron 1-2 (SEQ ID NO:1). Intron sequences are shown in lowercase letters with neighboring exon sequences shown in uppercase letters. The four sites for which engineered customized TAL effector repeat array domains were generated are underlined and numbered.

FIG. 3. Targeted demethylation at the human KLF4 gene by TAL-Tet1-CD fusion proteins harboring a GlysSer (SEQ ID NO:5) linker. Graphs show demethylation observed upon transfection of 4 different TAL-Tet1-CD fusion proteins into human K562 cells. Demethylation is calculated for each CpG by subtracting the % unmethylated CpGs in mock-transfected or GFP-transfected cells from the % unmethylated CpGs in cells in which TAL-Tet1-CD protein was expressed. Position of the TAL binding site is depicted with an arrow and direction of the arrow indicates N-terminal to C-terminal orientation of the protein. The specific TAL effector binding site targeted is indicated by a subscript, with TAL₁ referring to binding site #1, etc. and corresponding to the numbering of sites shown in FIG. 2. Where possible, error bars were calculated by pooling the standard error from the mean of both experimental and control replicates.

FIG. 4. Targeted demethylation at the human KLF4 gene using TAL-Tet1-CD fusion proteins harboring more extended linker variants. Graphs show demethylation observed upon transfection of 5 different TAL-Tet1-CD fusion proteins into human K562 cells (graphs show SEQ ID NO:6, SEQ ID NO:7, SEQ ID NO:8, SEQ ID NO:9, SEQ ID NO:10). Each fusion uses the same TAL DNA binding domain and Tet1-CD, but differs in the length and amino acid sequence used as a linker between the two domains. Demethylation is calculated for each CpG by subtracting the % unmethylated CpGs in mock-transfected or GFP-transfected cells from the % unmethylated CpGs in cells in which TAL-Tet1-CD protein was expressed. Position of the TAL binding site is depicted with an arrow and direction of the arrow indicates N-terminal to C-terminal orientation of the protein.

FIGS. 5A-C. Amino acid sequences of full length Tet1 (Tet1-FL; 5A: SEQ ID NO: 2), Tet1 catalytic domain (Tet1-CD; 5C: SEQ ID NO:4) and Tet1 protein with CXXC domain removed (Tet1-NZ; 5B: SEQ ID NO:3).

FIGS. 6A-D. A set of schematic illustrations showing Strategies for Tet1 Multimerization. A) Simple fusion of multiple Tet1 domains to one TALE DNA binding domain in either amino-terminal orientation, carboxy-terminal orientation, or both. B) Non-covalent DNA interaction schemes, showing either ligand-dependent binding (inducible multimerization) or direct binding through protein-protein interacting domains. C) Covalent DNA interaction scheme showing split-inteins, here designated as Int-N on the amino-terminal protein and Int-C on the carboxy-terminal protein, before and after splicing. D) An example of the use of several multimerization approaches at once showing the use of both split-inteins as well as multimerization of Tet1 domains in either amino-or cabroxy-terminal orientation with respect to the TALE DNA binding protein.

FIG. 7. List of human diseases and conditions associated with hypermethylation. Adapted from Table 1 of Rodenhiser and Mann, CMAJ 174(3):341-348 (2006). The references noted in the table are as follows:

1. Esteller M, Corn P G, Baylin S B, et al. Cancer Res 2001;61:3225-9.

2. Uhlmann K, Rohde K, Zeller C, et al. Int J Cancer 2003;106:52-9.

3. Gao Y, Guan M, Su B, et al. Clin Chim Acta 2004;349:173-9.

4. Bello M J, Alonso M E, Aminoso C, et al. Mutat Res 2004;554:23-32.

5. Mancini D N, Rodenhiser D I, Ainsworth P J, et al. Oncogene 1998;16:1161-9.

6. Szyf M, Pakneshan P, Rabbani S A. Biochem Pharmacol 2004;68:1187-97.

7. Widschwendter M, Jones P A. Oncogene 2002;21:5462-82.

8. Lea J S, Coleman R, Kurien A, et al. Am J Obstet Gynecol 2004; 190:674-9.

9. De Zhu J. Cell Res 2005; 15:272-80.

10. Chim C S, Kwong Y L, Fung T K, et al. Leuk Res 2004;28:379-85.

11. Esteller M, Silva J M, Dominquez G, et al. J Natl Cancer Inst 2000;92:564-9.

12. Li L C, Okino S T, Dahiya R. Biochim Biophys Acta 2004;1704:87-102.

13. Kurmasheva et al. Pediatr Blood Cancer 2005;44:328-37.

14. Sharma R P. Schizophr Res 2005;72:79-90.

15. Costa E, Chen Y, Davis J, et al. Mol Interv 2002;2:47-57.

16. Lund G, Anderson L, Lauria M, et al. J Biol Chem 2004;279:29147-54.

FIGS. 8A-C. Demethylation of human β-globin locus. A) Ratio of methylated to total CpGs at two CpGs located ˜300 bp upstream of the transcription start site in cells transfected with TALE-Tet1 proteins, GFP control (diamonds) or off-target control (squares). Arrows indicate location of TALE binding sites. B) Ratio of methylated to total CpGs at two CpGs located ˜100 bp downstream of the transcription start site in cells transfected with TALE-Tet1 proteins, GFP control (diamonds) or off-target control (squares). Arrows indicate location of TALE binding sites. C) Expression levels of β-globin mRNA in cells transfected with TALE-Tet1 proteins as determined by qRT-PCR. Fold activation is calculated relative to a GFP control. Error bars represent sem of three independent samples.

FIGS. 9A-B. Engineered Zinc Finger-Tet1 Fusion Proteins. A) Ratio of methylated to total CpGs at each position in the Klf4 locus in cells transfected with ZF-Tet1 proteins (diamonds and circles), GFP (diamonds) or an off-target control (squares). Arrows indicate the location of zinc finger binding sites. B) Ratio of methylated to total CpGs at each position in the B-globin locus in cells transfected with ZF-Tet1 proteins, GFP (squares) or an off-target control (VC117, squares). Arrows represent the location of ZF binding sites and are colored to correspond to their respective data points.

### DETAILED DESCRIPTION

Described herein are fusion proteins comprising a DNA-binding domain (i.e., an engineered custom DNA-binding domain), and a catalytic domain (from a different protein) comprising a sequence that catalyzes hydroxylation of methylated cytosines in DNA, with an optional linker between the two domains. As described herein, expression of a TAL effector repeat array-Tet1 (TAL-Tet1) fusion protein in human K562 cells results in demethylation of CpGs in close proximity to the target site bound by the TAL effector repeat array part of the protein.

Exemplified is a hybrid protein consisting of an engineered transcription activator-like (TAL) effector repeat array fused to either the full length Tet1 protein (Tet1-FL), the catalytic domain of Tet1 (Tet1-CD), or a truncated version of the Tet1-FL protein in which the putative native DNA-binding domain has been removed (Tet1-NZ). DNA-binding specificity is defined by the engineered TAL effector repeat array. These DNA-binding proteins can be engineered to bind to essentially any DNA sequence and published work from various labs, as well as published and unpublished work from our lab, has demonstrated that these customizable domains can efficiently target a variety of fused domains to specific genomic locations (Reyon et al., FLASH assembly of TALENs for high-throughput genome editing. Nat Biotechnol (2012).doi: 10.1038/nbt.2170; Moscou and Bogdanove, Science 326, 1501-1501 (2009); Boch et al., Science 326, 1509-1512 (2009); Miller et al., Nat Biotechnol 29, 143-148 (2010)). For example, engineered TAL effector repeat arrays have been fused to the cleavage domain of the FokI endonuclease as well as activators and repressors and act to target these domains to a user-defined sequence within the context of the genome.

### DNA-Binding Domains

The fusion proteins described herein can include any DNA Binding Domain (DBD) known in the art or engineered for a specific binding site. Exemplary DBDs include engineered or native TAL effector repeat arrays, engineered or native zinc fingers, homing meganucleases, chemical nucleases, and native DBDs.

### TAL Effector Repeat Arrays

TAL effectors of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity may be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

TALE proteins may be useful in research and biotechnology as targeted chimeric nucleases that can facilitate homologous recombination in genome engineering (e.g., to add or enhance traits useful for biofuels or biorenewables in plants). These proteins also may be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in USSN 61/610,212, and Reyon et al., Nature Biotechnology 30,460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci U S A 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci U S A 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

### Zinc Fingers

Zinc finger proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92:344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

Widespread adoption and large-scale use of zinc finger protein technology have been hindered by the continued lack of a robust, easy-to-use, and publicly available method for engineering zinc finger arrays. One existing approach, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34: W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

### Native DBDs

In some embodiments, a native DBD (e.g., a portion of a wild-type, non-engineered DNA binding protein that binds to a specific target sequence) can be used.

For example, the DBD from a transcription factor, nuclease, histone, telomerase, or other DNA binding protein can be used. Typically DBDs include a structure that facilitates specific interaction with a target nucleic acid sequence; common DBD structures include helix-turn-helix; zinc finger; leucine zipper; winged helix; winged helix turn helix; helix-loop-helix; and hmg-box. The native DBD can be from any organism. See, e.g., Kummerfeld & Teichmann, Nucleic Acids Res. 34 (Database issue): D74-81 (2006). The residues in a DNA binding protein that contact DNA, and thus form part of the DBD, can be determined empirically or predicted computationally, e.g., as described in Tjong and Zhou, Nucl. Acids Res. 35:1465-1477 (2007). A database of DNA binding proteins can be used to identify DNA binding proteins and DBDs for use in the present compositions and methods; see, e.g., Harrison, Nature, 353, 715-719 (1991); Karmirantzou and Hamodrakas, Protein Eng. 14(7): 465-472 (2001); Kumar et al., BMC Bioinformatics. 8:463 (2007); Kumar et al., J Biomol Struct Dyn. 26 (6): 679-86 (2009); Lin et al., PLoS One. 6 (9): e24756 (2011).

Where a native DBD is used in a fusion protein described herein, the catalytic domain is from a different protein.

### Homing Meganucleases

Meganucleases are sequence-specific endonucleases originating from a variety of organisms such as bacteria, yeast, algae and plant organelles. Endogenous meganucleases have recognition sites of 12 to 30 base pairs; customized DNA binding sites with 18 bp and 24 bp-long meganuclease recognition sites have been described, and either can be used in the present methods and constructs. See, e.g., Silva, G., et al., Current Gene Therapy, 11:11-27, (2011); Arnould et al., Journal of Molecular Biology, 355:443-58 (2006); Arnould et al., Protein Engineering Design & Selection, 24:27-31 (2011); and Stoddard, Q. Rev. Biophys. 38, 49 (2005); Grizot et al., Nucleic Acids Research, 38:2006-18 (2010).

### Chemical Nucleases

DNA binding domains from the so-called “chemical nucleases,” (Pingoud and Silva, Nat Biotechnol. 25:743-4 (2007)), e.g., triplex-forming oligonucleotides or peptide nucleic acids can also be utilized in the present compositions and methods; see, e.g., Schleifman et al., Methods Mol Biol. 2008;435:175-90; Arimondo et al., Mol Cell Biol. 2006 January; 26(1):324-33; Majumdar et al., J Biol Chem. 2008 Apr. 25; 283(17):11244-52; Simon et al., Nucleic Acids Res. 2008 June; 36(11):3531-8; or Eisenschmidt et al., Nucleic Acids Res. 2005; 33(22):7039-47.

### Catalytic Domains

The fusion proteins include a catalytic domain comprising a sequence that catalyzes hydroxylation of methylated cytosines in DNA. Exemplary proteins include the Ten-Eleven-Translocation (TET) 1-3 family, enzymes that converts 5-methylcytosine (5-mC) to 5-hydroxymethylcytosine (5-hmC) in DNA.

Sequences for human TET1-3 are known in the art and are shown in the following table:

GenBank Accession Nos.

| Gene | Amino Acid | Nucleic Acid |
|---|---|---|
| TET1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

*Variant (1) represents the longer transcript and encodes the longer isoform (a). Variant (2) differs in the 5′ UTR and in the 3′ UTR and coding sequence compared to variant 1. The resulting isoform (b) is shorter and has a distinct C-terminus compared to isoform a.

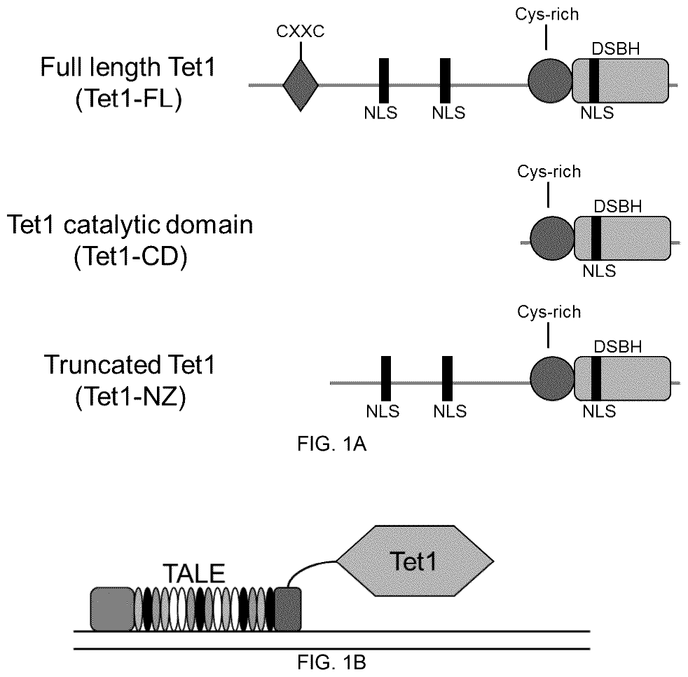

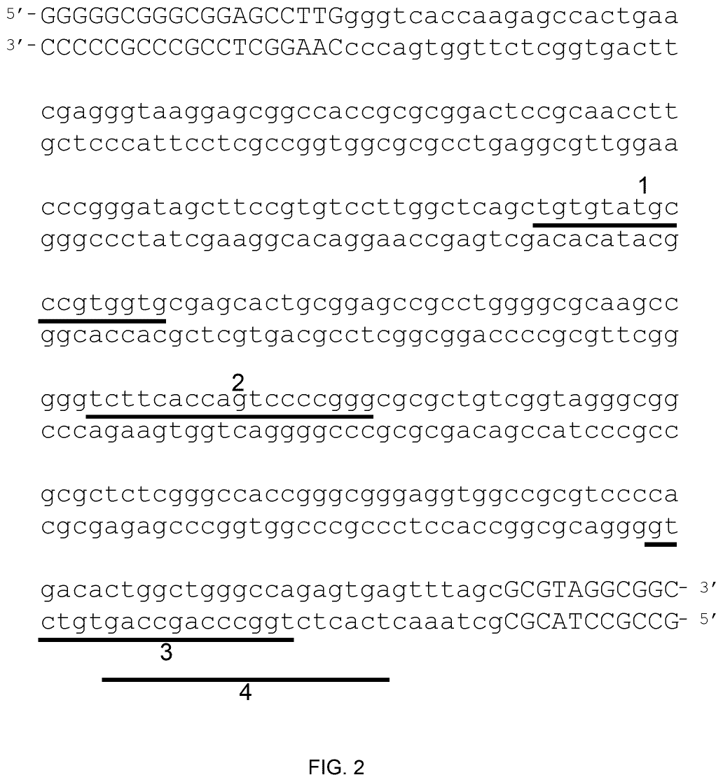

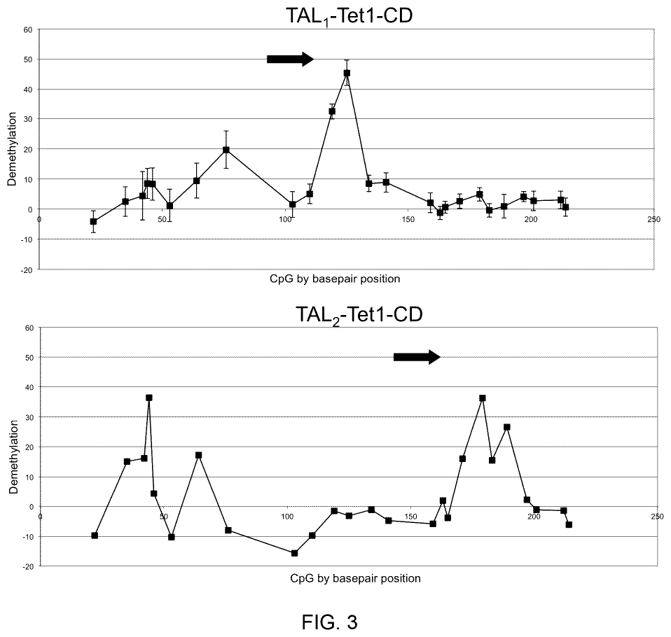

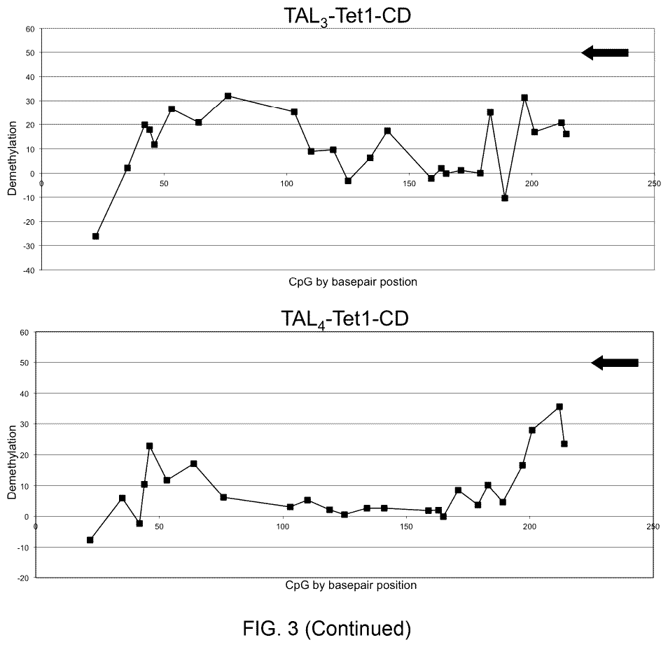

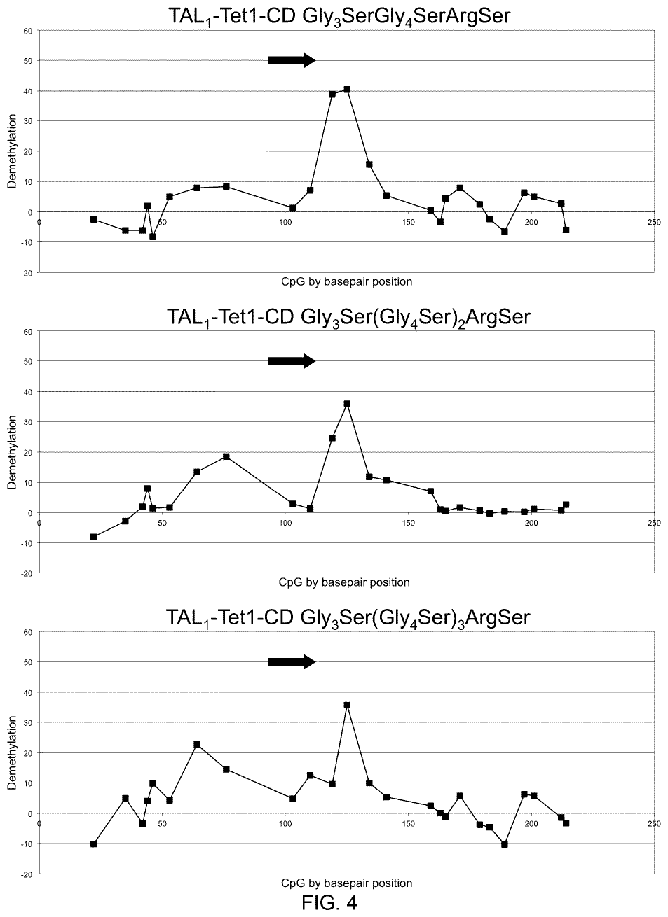

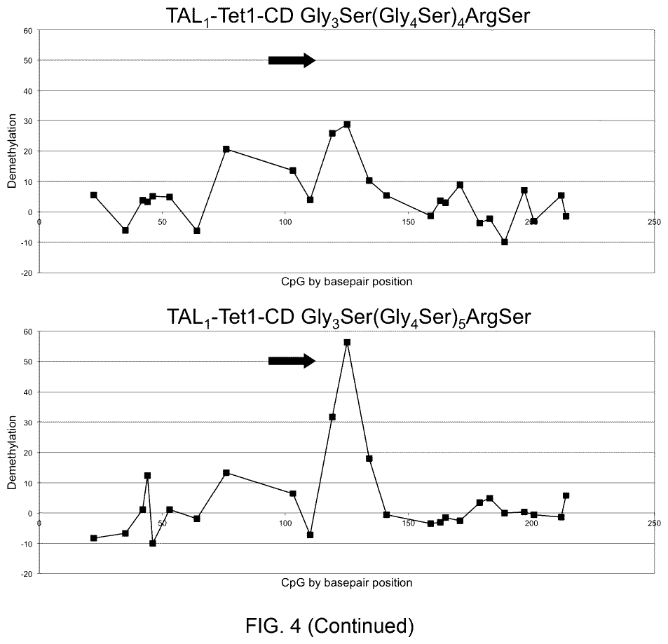

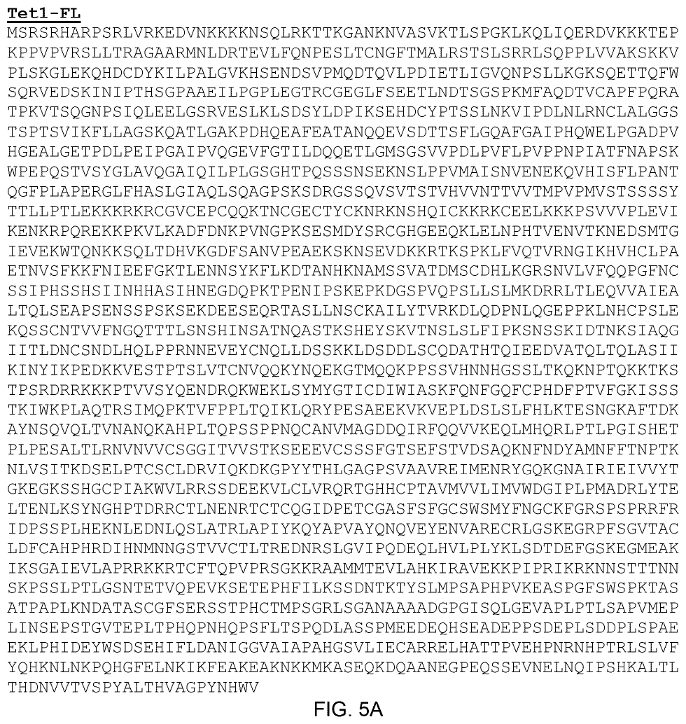

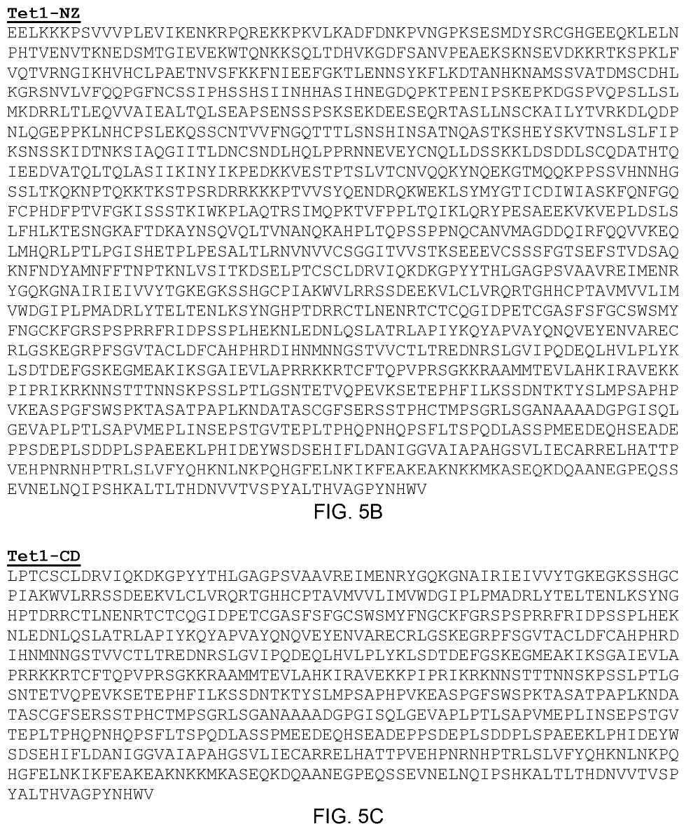

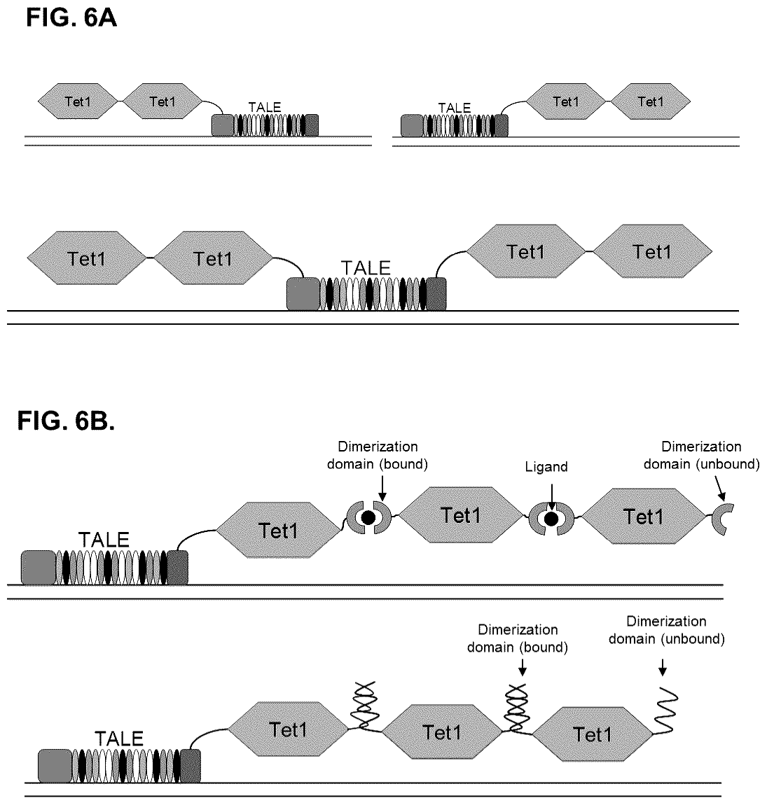

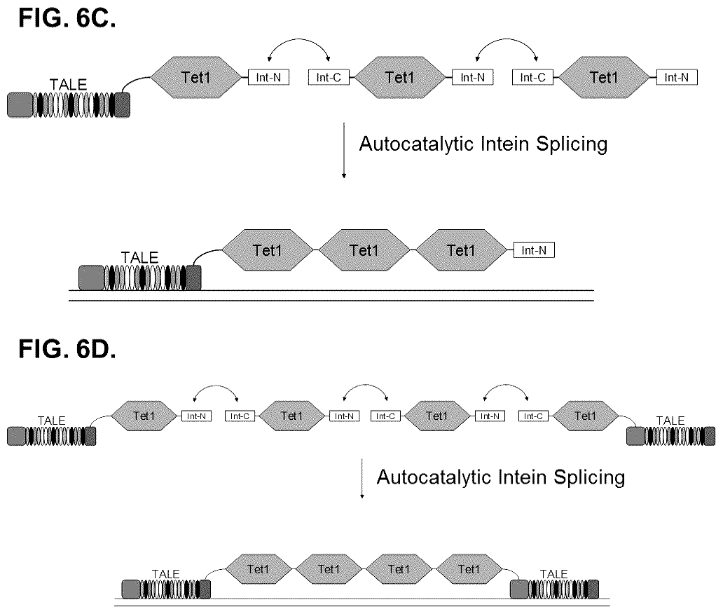

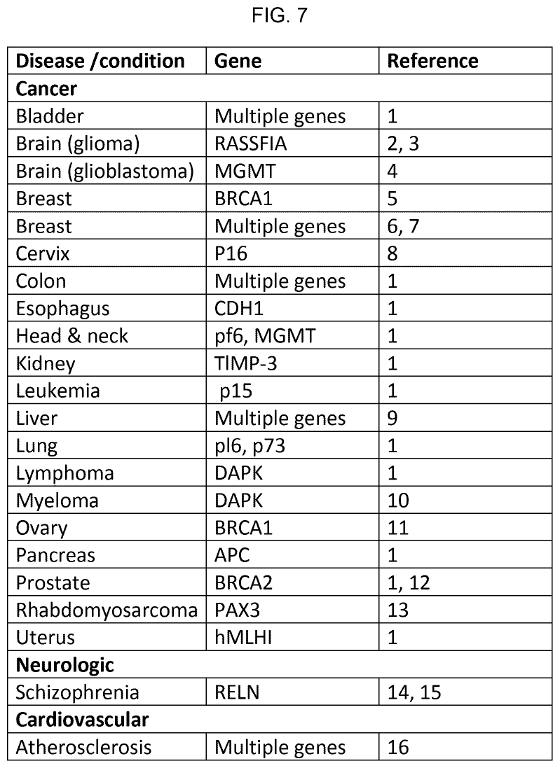

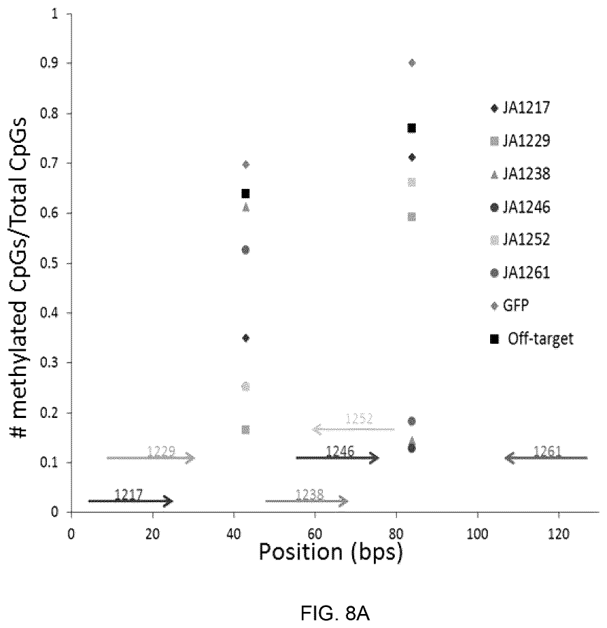

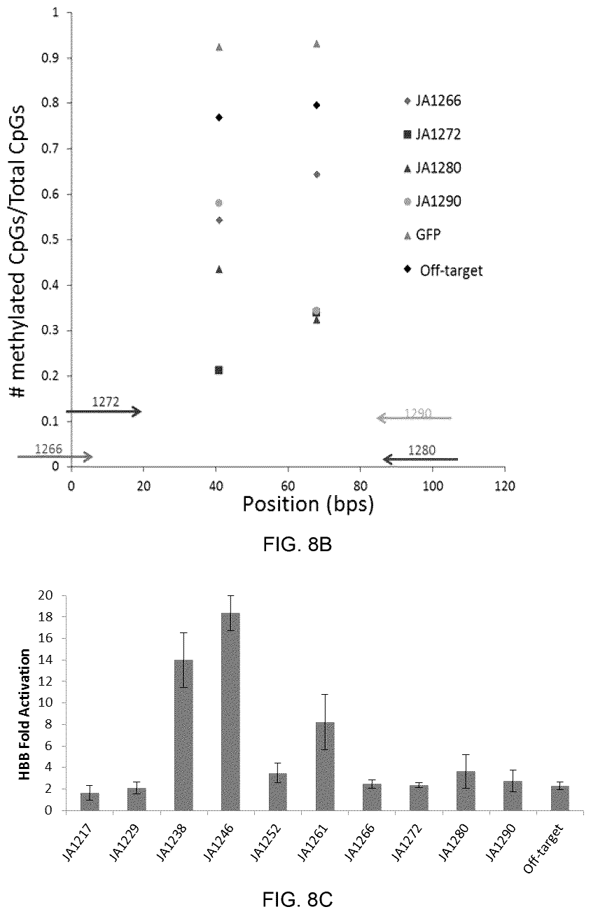

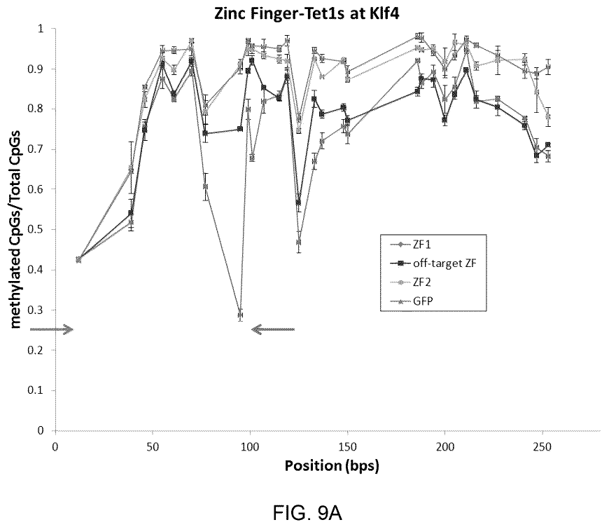

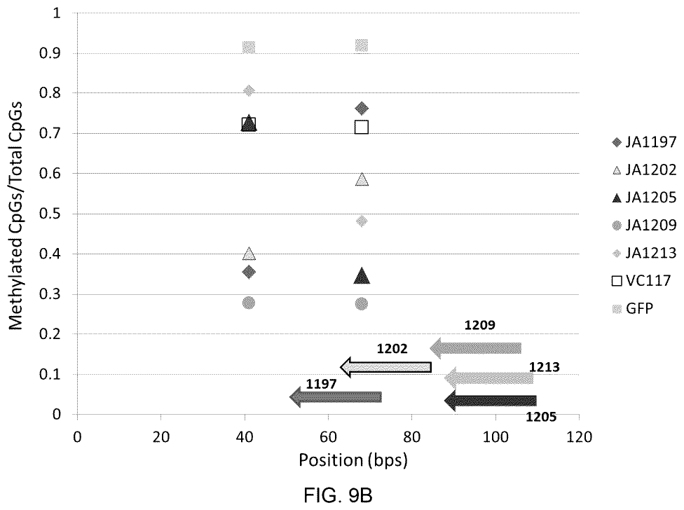

In some embodiments, all or part of the full-length sequence of the catalytic domain can be included, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1A and 1B of Iyer et al., Cell Cycle. 2009 Jun. 1;8(11):1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

### Construction of Fusion Proteins

To generate a functional recombinant protein, the DNA binding domain is fused to at least one catalytic domain. Fusing catalytic domains to DBD to form functional fusion proteins involves only routine molecular biology techniques that are commonly practiced by those of skill in the art, see for example, U.S. Pat. Nos. 6,511,808, 6,013,453, 6,007,988, 6,503,717 and U.S. patent application 2002/0160940). Catalytic domains can be associated with the DBD domain at any suitable position, including the C- or N-terminus of the DBD. In some embodiments, the fusion proteins include multiple catalytic domains, e.g., on one or both ends of the DBD, e.g., concatenated together with an optional intervening linker; thus there can be one or more catalytic domains on each end of the DBD.

Alternatively, the catalytic domains, e.g., Tet1 units, could be multimerized through specific constitutive or inducible protein-protein interaction domains (such as leucine zipper domains or ClonTech's iDimerize system, homodimerization and heterodimerization systems and ligands (e.g. AP20187, AP21967) which were previously provided by ARIAD under the brand name ARGENT. The B/B Homodimerizer (AP20187) induces dimerization of two proteins that each contain the DmrB homodimerization domain (see table below explaining domain nomenclature). The A/C Heterodimerizer (AP21967) induces dimerization of a protein possessing the DmrA domain and a second protein containing the DmrC domain. The D/D Solubilizer (alternative to AP21998) induces dissociation/disaggregation of proteins possessing DmrD domains. DmrD causes automatic self-association of proteins fused to it; see, e.g., Burnett et al., J. Leukoc. Biol. 75(4):612-623 (2004); Freeman et al., Cancer Res. 63(23) 8256-8563 (2003); Castellano et al., Curr. Biol. 9(7): 351-360 (1999); Crabtree and Schreiber, Trends Biochem. Sci. 21(11): 418-422 (1996); Graef et al., Embo. J. 16(18): 5618-5628 (1997); Muthuswamy et al., Mol. Cell. Biol. 19(10): 6845-6857 (1999)), Alternatively, multimerization could be achieved through the use of split-inteins, a class of autocatyltic intein peptides that allow for the seamless covalent splicing of two separate proteins in a predictable and efficient manner (d'Avignon, et al., Biopolymers. 2006 Oct. 15;83(3):255-67; Zitzewitz, et al., Biochemistry. 1995 Oct. 3;34(39):12812-9; Li et al., Hum Gene Ther. 2008 September;19(9):958-64). Both the protein-protein interaction and intein approaches could be optimized to produce very long multimerized strings of catalytic domains. FIGS. 6A-D show exemplary schemes for multimerization.

### Methods of Use of the Fusion Proteins

The fusion proteins designed and produced using the methods described herein can be used to perform any function where it is desired to demethylate a specific DNA sequence. The fusion proteins can be useful for the treatment of disease; for example, the fusion proteins can be targeted to a hypermethylated region of a gene listed in Table 1 of Rodenhiser and Mann, CMAJ 174(3):341-348 (2006) as hypermethylated in a disease, which is reproduced as FIG. 7 hereto, or a reference set forth therein, or for otherwise altering the structure or function of a given gene in vivo. The fusion proteins of the present invention are also useful as research tools, for example, in performing either in vivo or in vitro functional genomics studies (see, for example, U.S. Pat. No. 6,503,717 and U.S. patent application 2002/0164575).

### Polypeptide Expression Systems

In order to use the fusion proteins described, it may be desirable to express the engineered proteins from a nucleic acid that encodes them. This can be performed in a variety of ways. For example, the nucleic acid encoding the fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or for production of the fusion protein. The nucleic acid encoding the fusion protein can also be cloned into an expression vector, for administration to a plant cell, animal cell, preferably a mammalian cell or a human cell, fungal cell, bacterial cell, or protozoan cell.

To obtain expression, the fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered TALE repeat protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of the fusion protein nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein, “MBP.” Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, NUCLEOFECTION, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

### Use of Fusion Proteins in Gene Therapy

The fusion proteins described herein can be used to regulate gene expression or alter gene sequence in gene therapy applications in the same. See for example U.S. Pat. No. 6,511,808, U.S. Pat. No. 6,013,453, U.S. Pat. No. 6,007,988, U.S. Pat. No. 6,503,717, U.S. patent application 2002/0164575, and U.S. patent application 2002/0160940. The methods can include administering one or more of the fusion proteins described herein targeted to one or more genes. Since methylation of many CpGs across hundreds of basepairs of DNA in promoters or imprinted regions can influence gene expression (Han et al., Mol Cell Biol. 2008 February;28 (3): 1124-35, Mabaera, et al., Blood. 2007 Aug. 15;110(4):1343-52), it may be desirable to reduce methulation across longer sequences. If a larger region of a gene is desired to be demethylated, a plurality of fusion proteins that all target positions on the same gene, e.g., positions within about 1000, 500, 300, 250, 100, 50, 40, 30, or 20 bp of the site to be demethylated, can be administered. Alternatively or in addition, one or a plurality of fusion proteins that are multimerized as described herein (see, e.g., FIGS. 6A-D) can be administered.

Conventional viral and non-viral based gene transfer methods can be used to introduce nucleic acids encoding the fusion protein into mammalian cells or target tissues. Such methods can be used to administer nucleic acids encoding fusion proteins to cells in vitro. Preferably, the nucleic acids encoding the fusion proteins are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, 1992, Science, 256:808-813; Nabel & Felgner, 1993, TIBTECH, 11:211-217; Mitani & Caskey, 1993, TIBTECH, 11:162-166; Dillon, 1993, TIBTECH, 11:167-175; Miller, 1992, Nature, 357:455-460; Van Brunt, 1988, Biotechnology, 6:1149-54; Vigne, 1995, Restorat. Neurol. Neurosci., 8:35-36; Kremer & Perricaudet, 1995, Br. Med. Bull., 51:31-44; Haddada et al., in Current Topics in Microbiology and Immunology Doerfler and Bohm (eds) (1995); and Yu et al., 1994, Gene Ther., 1:13-26.

Methods of non-viral delivery of nucleic acids encoding the fusion proteins include lipofection, microinjection, biolistics, virosomes, liposomes, immunoliposomes, polycation or lipid: nucleic acid conjugates, naked DNA or RNA, artificial virions, and agent-enhanced uptake of DNA or RNA. Lipofection is described in e.g., U.S. Pat. Nos. 5,049,386, 4,946,787; and 4,897,355) and lipofection reagents are sold commercially (e.g., Transfectam3 and Lipofectin3). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid: nucleic acid complexes, including targeted liposomes such as immunolipid complexes, is well known to one of skill in the art (see, e.g., Crystal, 1995, Science, 270:404-410; Blaese et al., 1995, Cancer Gene Ther., 2:291-297; Behr et al., 1994, Bioconjugate Chem. 5:382-389; Remy et al., 1994, Bioconjugate Chem., 5:647-654; Gao et al., Gene Ther., 2:710-722; Ahmad et al., 1992, Cancer Res., 52:4817-20; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding the fusion proteins takes advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of fusion proteins could include retroviral, lentivirus, adenoviral, adeno-associated, Sendai, and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992, J. Virol., 66:2731-39; Johann et al., 1992, J. Virol., 66:1635-40; Sommerfelt et al., 1990, Virololgy, 176:58-59; Wilson et al., 1989, J. Virol., 63:2374-78; Miller et al., 1991, J. Virol., 65:2220-24; WO 94/26877).

In applications where transient expression of the fusion protein is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987, Virology 160:38-47; U.S. Pat. No. 4,797,368; WO 93/24641; Kotin, 1994, Hum. Gene Ther., 5:793-801; Muzyczka, 1994, J. Clin. Invest., 94:1351). Construction of recombinant AAV vectors are described in a number of publications, including U.S. Pat. No. 5,173,414; Tratschin et al., 1985, Mol. Cell. Biol. 5:3251-60; Tratschin et al., 1984, Mol. Cell. Biol., 4:2072-81; Hermonat & Muzyczka, 1984, Proc. Natl. Acad. Sci. USA, 81:6466-70; and Samulski et al., 1989, J. Virol., 63:3822-28.

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., 1995, Blood, 85:3048; Kohn et al., 1995, Nat. Med., 1:1017; Malech et al., 1997, Proc. Natl. Acad. Sci. USA, 94:12133-38). PA317/pLASN was the first therapeutic vector used in a gene therapy trial. (Blaese et al., 1995, Science, 270:475-480). Transduction efficiencies of 50% or greater have been observed for MFG-S packaged vectors (Ellem et al., 1997, Immunol Immunother., 44:10-20; Dranoffet al., 1997, Hum. Gene Ther., 1:111-112).

Recombinant adeno-associated virus vectors (rAAV) are a promising alternative gene delivery systems based on the defective and nonpathogenic parvovirus adeno-associated type 2 virus. Typically, the vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., 1998, Lancet, 351:1702-1703; Kearns et al., 1996, Gene Ther., 9:748-55).

Replication-deficient recombinant adenoviral vectors (Ad) are predominantly used for colon cancer gene therapy, because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., 1998, Hum. Gene Ther. 7:1083-89). Additional examples of the use of adenovirus vectors for gene transfer in clinical trials include Rosenecker et al., 1996, Infection, 24:15-10; Sterman et al., 1998, Hum. Gene Ther., 9:7 1083-89; Welsh et al., 1995, Hum. Gene Ther., 2:205-218; Alvarez et al., 1997, Hum. Gene Ther. 5:597-613; Topf et al., 1998, Gene Ther., 5:507-513;

Sterman et al., 1998, Hum. Gene Ther., 7:1083-89.

Packaging cells are used to form virus particles that are capable of infecting a host cell. Such cells include 293 cells, which package adenovirus, and Ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, Proc. Natl. Acad. Sci. USA, 92:9747-51, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or stem cells (e.g., universal donor hematopoietic stem cells, embryonic stem cells(ES), partially differentiated stem cells, non-pluripotent stem cells, pluripotent stem cells, induced pluripotent stem cells (iPS cells) (see e.g., Sipione et al., Diabetologia, 47:499-508, 2004)), followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with nucleic acid (gene or cDNA), encoding the fusion protein, and re-infused back into the subject organism (e.g., patient). Various cell types suitable for ex vivo transfection are well known to those of skill in the art (see, e.g., Freshney et al., Culture of Animal Cells, A Manual of Basic Technique (5th ed. 2005)) and the references cited therein for a discussion of how to isolate and culture cells from patients).

In one embodiment, stem cells (e.g., universal donor hematopoietic stem cells, embryonic stem cells(ES), partially differentiated stem cells, non-pluripotent stem cells, pluripotent stem cells, induced pluripotent stem cells (iPS cells) (see e.g., Sipione et al., Diabetologia, 47:499-508, 2004)) are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma and TNF-alpha are known (see Inaba et al., 1992, J. Exp. Med., 176:1693-1702).

Stem cells can be isolated for transduction and differentiation using known methods. For example, stem cells can be isolated from bone marrow cells by panning the bone marrow cells with antibodies which bind unwanted cells, such as CD4+ and CD8+ (T cells), CD45+ (panB cells), GR-1 (granulocytes), and 1ad (differentiated antigen presenting cells) (see Inaba et al., 1992, J. Exp. Med., 176:1693-1702).

Vectors (e.g., retroviruses, adenoviruses, liposomes, etc.) containing nucleic acids encoding the fusion protein can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Alternatively, stable formulations of the fusion protein can also be administered.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington: The Science and Practice of Pharmacy, 21st ed., 2005).

### Delivery Vehicles

An important factor in the administration of polypeptide compounds, such as the fusion proteins of the present invention, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as fusion protein across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, 1996, Curr. Opin. Neurobiol., 6:629-634). Another subsequence, the h (hydrophobic) domain of signal peptides, was found to have similar cell membrane translocation characteristics (see, e.g., Lin et al., 1995, J. Biol. Chem., 270:14255-58).

Examples of peptide sequences that can be linked to a protein, for facilitating uptake of the protein into cells, include, but are not limited to: peptide fragments of the tat protein of HIV (Endoh et al., 2010, Methods Mol. Biol., 623:271-281; Schmidt et al., 2010, FEBS Lett., 584:1806-13; Futaki, 2006, Biopolymers, 84:241-249); a 20 residue peptide sequence which corresponds to amino acids 84-103 of the p16 protein (see Fahraeus et al., 1996, Curr. Biol., 6:84); the third helix of the 60-amino acid long homeodomain of Antennapedia (Derossi et al., 1994, J. Biol. Chem., 269:10444); the h region of a signal peptide, such as the Kaposi fibroblast growth factor (K-FGF) h region (Lin et al., supra); or the VP22 translocation domain from HSV (Elliot & O'Hare, 1997, Cell, 88:223-233). See also, e.g., Caron et al., 2001, Mol Ther., 3:310-318; Langel, Cell-Penetrating Peptides: Processes and Applications (CRC Press, Boca Raton FL 2002); El-Andaloussi et al., 2005, Curr. Pharm. Des., 11:3597-3611; and Deshayes et al., 2005, Cell. Mol. Life Sci., 62:1839-49. Other suitable chemical moieties that provide enhanced cellular uptake may also be chemically linked to Fusion proteins described herein.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., 1993, J. Biol. Chem., 268:3334-41; Perelle et al., 1993, Infect. Immun., 61:5147-56; Stenmark et al., 1991, J. Cell Biol., 113:1025-32; Donnelly et al., 1993, Proc. Natl. Acad. Sci. USA, 90:3530-34; Carbonetti et al., 1995, Abstr. Annu. Meet. Am. Soc. Microbiol. 95:295; Sebo et al., 1995, Infect. Immun., 63:3851-57; Klimpel et al., 1992, Proc. Natl. Acad. Sci. USA, 89:10277-81; and Novak et al., 1992, J. Biol. Chem., 267:17186-93).

Such subsequences can be used to translocate fusion proteins across a cell membrane. The fusion proteins can be conveniently fused to or derivatized with such sequences. Typically, the translocation sequence is provided as part of a fusion protein. Optionally, a linker can be used to link the fusion protein and the translocation sequence. Any suitable linker can be used, e.g., a peptide linker.

The fusion protein can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., the fusion protein.

The liposome fuses with the plasma membrane, thereby releasing the compound into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (e.g., the fusion protein or a nucleic acid encoding the same) at the target tissue or cell. For systemic or tissue specific delivery, this can be accomplished, for example, in a passive manner wherein the liposome bilayer degrades over time through the action of various agents in the body. Alternatively, active compound release involves using an agent to induce a permeability change in the liposome vesicle. Liposome membranes can be constructed so that they become destabilized when the environment becomes acidic near the liposome membrane (see, e.g., Proc. Natl. Acad. Sci. USA, 84:7851 (1987); Biochemistry, 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise the fusion protein and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., 1980, Annu. Rev. Biophys. Bioeng., 9:467, U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication. No. WO 91/17424, Deamer & Bangham, 1976, Biochim. Biophys. Acta, 443:629-634; Fraley, et al., 1979, Proc. Natl. Acad. Sci. USA, 76:3348-52; Hope et al., 1985, Biochim. Biophys. Acta, 812:55-65; Mayer et al., 1986, Biochim. Biophys. Acta, 858:161-168; Williams et al., 1988, Proc. Natl. Acad. Sci. USA, 85:242-246; Liposomes (Ostro (ed.), 1983, Chapter 1); Hope et al., 1986, Chem. Phys. Lip., 40:89; Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it is desirable to target liposomes using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., 1990, J. Biol. Chem., 265:16337-42 and Leonetti et al., 1990, Proc. Natl. Acad. Sci. USA, 87:2448-51).

### Dosages

For therapeutic applications, the dose of the fusion protein to be administered to a patient can be calculated in a similar way as has been described for zinc finger proteins, see for example U.S. Pat. No. 6,511,808, U.S. Pat. No. 6,492,117, U.S. Pat. No. 6,453,242, U.S. patent application 2002/0164575, and U.S. patent application 2002/0160940. In the context of the present disclosure, the dose should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy, specificity, and Kp of the particular fusion protein employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

### Pharmaceutical Compositions and Administration

Appropriate pharmaceutical compositions for administration of the fusion proteins of the present invention can be determined as described for zinc finger proteins, see for example U.S. Pat. No. 6,511,808, U.S. Pat. No. 6,492,117, U.S. Pat. No. 6,453,242, U.S. patent application 2002/0164575, and U.S. patent application 2002/0160940. Fusion proteins, and expression vectors encoding fusion proteins, can be administered directly to the patient for modulation of methylation patterns, e.g., and gene expression, and for therapeutic or prophylactic applications, for example, for treatment of diseases listed in FIG. 7 as associated with hypermethylation, including cancer (e.g., bladder, brain (e.g., glioma, or glioblastoma), breast, cervical, colon, colorectal, esophagus, head/neck, kidney, leukemia, liver, lung, lymphoma, myeloma, ovary, pancreas, prostate, rhabdomyosarcoma, and uterus cancer); schizophrenia; memory formation; and atherosclerosis. Thus the methods can include identifying a subject who has a disease associated with hypermethylation (e.g., optionally including obtaining a sample and detecting methylation of DNA, e.g., of the associated gene listed in FIG. 7, and selecting the subject if their DNA is hypermethylated), and administering a therapeutically effective amount of a fusion protein, or a nucleic acid encoding a fusion protein, as described herein, to the subject.

Administration of therapeutically effective amounts is by any of the routes normally used for introducing Fusion proteins into ultimate contact with the tissue to be treated. The fusion proteins are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington: The Science and Practice of Pharmacy, 21st ed., 2005).

The fusion proteins, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

### EXAMPLES

The invention is further described in the following examples, which do not limit the scope of the invention described in the claims.

### Example 1. Generation of TAL-Tet1 Fusion Proteins to Specific Genomic Loci in the Endogenous Human KLF4 Gene

Plasmids expressing three different versions of the human Tet1 protein were constructed based on the predicted domain architecture described by Tahiliani et al. (Science 324, 930-935 (2009)) (FIG. 1A). Tet1-FL is the full length Tet1 protein, while Tet1-CD comprises only the catalytic domain of the protein, including the Cys-rich region and the double-stranded β-helix domain (FIGS. 5A, 5B and 5C). Tet1-NZ is an intermediate truncation variant that lacks a CXXC-type zinc-binding domain, which may be responsible for directing DNA-binding of the endogenous protein (Zhang et al., Cell Res 20, 1390-1393 (2010); Xu et al., Mol. Cell 42, 451-464 (2011); FIGS. 5A, 5B and 5C). Note that Tet1-NZ maintains more of the native protein than Tet1-CD alone. Briefly, DNA fragments encoding full-length Tet1 (Tet1-FL; amino acids 1-2136) and Tet1 catalytic domain (Tet1-CD; amino acids 1418-2136) were synthesized in 8 pieces (Integrated DNA Technologies) and iteratively cloned together using standard restriction digest/ligation based cloning.

These fragments encoding Tet1-FL and Tet1-CD were then cloned into a plasmid vector that enables their expression as fusions to the carboxy-terminal end of four different engineered TAL repeat array protein domains designed to bind different 18 bp target sites within the second intron of the human Klf4 gene (see FIG. 2). The linker sequences used to join the Tet1 protein or Tet1 protein fragments to the TAL effector repeat arrays were composed of one of the following linkers: Gly₃Ser (SEQ ID NO:5), Gly₃SerGly₄SerArgSer (SEQ ID NO:6), Gly₃Ser(Gly₄Ser)₂ArgSer (SEQ ID NO:7), Gly₃Ser(Gly₄Ser)₃ArgSer (SEQ ID NO:8), Gly₃Ser(Gly₄Ser)₄ArgSer (SEQ ID NO:9), or Gly₃Ser(Gly₄Ser)₅ArgSer (SEQ ID NO:10).

The final expression plasmid placed the fusion proteins under the transcriptional control of a constitutively active EF1α promoter. Tet1-NZ was constructed by removing the CXXC domain located at amino acids 584-624 of the full length protein. This was achieved by using tailed PCR of a small segment of Tet1-FL that placed a BamHI site immediately after the CXXC domain and included a PmlI site upstream of the CXXC domain that is unique in the Tet1-FL expression plasmid. This PCR product was then cloned into the Tet1-FL expression plasmid using a BamHI/PmlI double digest of both the PCR product and the Tet1-FL plasmid followed by ligation and transformation.

The four genomic loci chosen to target for demethylation (and for which TAL effector repeat arrays were designed) were selected based on several criteria. All four loci lie within intron 1-2 of the human KLF4 gene, which exhibits marks of open chromatin based on data from the ENCODE project (as accessed through the UCSC

Genome Browser), suggesting its likely accessibility for binding by TAL effector DNA binding domains. This intron was also attractive because it harbors multiple CpG diresidues that are methylated in K562 cells but that are unmethylated in human embyronic stem cells, suggesting that methylation of these cytosines is not necessary for cell survival or viability.

TAL effector repeat arrays were engineered using the previously described FLASH assembly method (Reyon et al. FLASH assembly of TALENs for high-throughput genome editing. Nat Biotechnol (2012).doi:10.1038/nbt.2170). DNA fragments encoding assembled arrays were cloned into expression constructs containing amino acids 153 to 288 from the TALE13 protein and the +95 C-terminal domain, comprised of amino acids 715 to 809 from the C-terminal end of TALE13 (Miller et al., Nat Biotechnol 29, 143-148 (2010)).

### Example 2. Site-Specific Demethylation of CpGs in the Human KLF4 Locus Mediated by TAL-Tet1 Fusion Proteins

Human K562 cells were cultured in RPMI 1640 medium (Gibco) with 10% HI-FBS (Gibco), 1% GLUTAMAX (Gibco) and 1% Pen/Strep (Gibco), and were transfected with plasmids encoding various TAL-Tet1-CD fusion proteins harboring the Gly₃Ser (SEQ ID NO:5) linker by nucleofecting 1×10⁶ dividing cells with 10 μg of DNA in 100 μl of Kit V solution (Lonza) using program T-016 on the Nucleofector 2b Device (Lonza). Nucleofected cells were incubated in 6-well plates at 37° C. for 4 days following NUCLEOFECTION. Genomic DNA was harvested 4 days post-transfection. Bisulfite DNA sequencing of KLF4 intron 1-2 from these transfected cell populations was performed as follows. Genomic DNA was isolated from transfected cells using the Qiagen Blood Mini kit. 200-1000 ng of genomic DNA was bisulfite treated using either the EZ DNA Methylation Kit (Zymo), EZ DNA METHYLATION-LIGHTNING Kit (Zymo), or Cells-to-CpG Bisulfite Conversion Kit (Applied Biosystems) following recommended protocols. PCR amplification of Bis-DNA was performed using ACCUPRIME HiFi Taq DNA polymerase system (Invitrogen). PCR products were TA-cloned using the Original TA Cloning protocol (Invitrogen) or the pGEM-T Easy Vector System (Promega) and transformed into either Top10 or XL1-BLUE competent cells. Individual clones were miniprepped and sequenced and sequences were analyzed using the BISMA software available online at (Rohde et al., BMC Bioinformatics 2010, 11:230), using default parameters.

The results revealed robust demethylation at CpGs positioned adjacent to the site bound by each of these TAL-Tet1-CD fusion proteins. FIG. 3 shows the rates of demethylation for CpGs near the four different TAL binding sites. TAL-Tet1-CD protein targeted to sitel showed the highest levels of demethylation with 32.5% and 45.4% demethylation of CpGs located 9 and 15 bp downstream of the target binding site, respectively. TAL-Tet1-CD protein targeted to site 2 showed no demethylation of CpGs located 1 and 3 bp downstream of the binding site but did show 36.5% and 28.2% demethylation at CpGs located 17 and 27 bp downstream, respectively. TAL-Tet1 protein targeted to site 3 showed several greater than 25% demethylation at CpGs located 22, 36, 116 and 143 bp downstream, and a TAL-Tet1-CD protein targeted to site 4 showed a 37% demethylation at a CpG located 13 bp downstream but failed to show significant demethylation at any other CpGs.

Overall, this demonstrates the ability of engineered TAL-Tet1-CD fusion proteins harboring a Gly₃Ser (SEQ ID NO:5) linker to induce 30-50% demethylation of CpGs located approximately 10-30 bp downstream of the TAL binding site. (Note for all of these cases, “downstream” refers to bases located 3′ to the last base of the binding site contacted by the carboxy-terminal TAL effector repeat in the fusion protein.) TAL-Tet1-CD fusion proteins harboring more extended linkers (Gly₃SerGly₄SerArgSer (SEQ ID NO:6), Gly₃Ser (Gly₄Ser) 2ArgSer (SEQ ID NO:7), Gly₃Ser (Gly₄Ser) 3ArgSer (SEQ ID NO:8), Gly₃Ser (Gly₄Ser) 4ArgSer (SEQ ID NO:9), or Gly₃Ser (Gly₄Ser) 5ArgSer (SEQ ID NO:10) also showed similar levels of demethylation (FIG. 4).

### Example 3. Engineered TALE-Tet1 Proteins Targeted to the Human HBB Locus

Published work has identified a small number of CpGs in the human β-globin and γ-globin promoters which are differentially methylated in fetal liver and adult bone marrow cells and has suggested that methylation of these CpGs may dictate expression of these genes during development (Mabaera et al., Blood. 2007 Aug. 15;110 (4): 1343-52). TALE arrays targeted to sequences near these differentially methylated CpGs at the human β-globin locus were engineered: four TALEs targeted to a set of two CpGs ˜100 bp downstream of the transcription start site and six TALEs targeted to a set of two CpGs ˜300 bp upstream of the start site.

Human K562 cells were cultured in RPMI 1640 medium (Gibco) with 10% HI-FBS (Gibco), 1% GLUTAMAX (Gibco) and 1% Pen/Strep (Gibco), and were transfected with plasmids encoding various TAL-Tet1-CD fusion proteins harboring the Gly₃Ser (SEQ ID NO:5) linker by nucleofecting 1×10⁶ dividing cells with 10 μg of DNA in 100 μl of Kit V solution (Lonza) using program T-016 on the Nucleofector 2b Device (Lonza). Nucleofected cells were incubated in 6-well plates at 37° C. for 4 days following NUCLEOFECTION. Genomic DNA was harvested 4 days post-transfection. Bisulfite DNA sequencing of the HBB locus from these transfected cell populations was performed as follows. Genomic DNA was isolated from transfected cells using the Qiagen Blood Mini kit. 200-1000 ng of genomic DNA was bisulfite treated using either the EZ DNA Methylation Kit (Zymo), EZ DNA METHYLATION-LIGHTNING Kit (Zymo), or Cells-to-CpG Bisulfite Conversion Kit (Applied Biosystems) following recommended protocols. PCR amplification of Bis-DNA was performed using Pyromark PCR kit (Qiagen). Illumina adapters and barcodes were added by PCR with Phusion High-Fidelity PCR enzyme (NEB) and amplicons were sequenced on an Illumina MISEQ system. Total RNA was isolated from the same cells with the PURELINK RNA mini kit (Ambion) according to manufacturer's instructions. Reverse transcription was performed with the SUPERSCRIPTIII RT kit (Invitrogen) and TAQMAN assays were run on an Applied Biosystems 7500Fast Real Time PCR machine.

All TALE-Tet1 fusions were able to reduce methylation of CpGs located downstream of the TALE binding site, albeit with varying efficiencies (FIGS. 8A-B). Interestingly, qRT-PCR analysis of β-globin levels showed increased expression only in those cells transfected with one of three TALE-Tet1 proteins targeting the CpG 266 bp upstream of the transcription start site (FIG. 8C). Thus, while all of the TALE-Tet1 proteins were able to catalyze demethylation of nearby CpGs, only demethylation of this single CpG causes increased expression of the β-globin gene.

These results demonstrate that the TALE-Tet1 fusion proteins can be used to demethylate specific CpG residues in a gene promoter and thereby upregulate expression of that gene in living cells.

### Example 4. Engineered ZF-Tet1 Fusion Proteins

In addition to generating TALE-Tet1 fusions, Zinc Finger (ZF)-Tet1 fusion proteins were also engineered. Six-finger zinc finger proteins were assembled from two-finger ZF units derived from OPEN-selected proteins. Two ZF-Tet1 fusions targeted to the Klf4 locus and five targeted to the β-globin locus were engineered.

Upon transfection into K562 cells, one of the Klf4 ZF-Tet1 proteins was able to demethylate CpGs located 6 and 24 bp downstream of the ZF binding site by 68 and 25%, respectively (FIG. 9A).

Additionally, five ZF-Tet1 proteins targeted to the two CpGs located ˜100 bp downstream of the β-globin transcription start site were engineered. All five of these proteins were able to induce demethylation of CpGs located downstream of the ZF binding site, albeit with varying efficiencies (FIG. 9B). This data demonstrates that the catalytic domain of Tet1 can be fused to alternate DNA-binding domains in order to generate site-specific DNA demethylation enzymes, resulting upregulation of expression of targeted genes in living cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-22. (canceled)

23. A fusion protein comprising
an engineered transcription activator-like (TAL) effector repeat array or a zinc finger protein DNA-binding domain that binds specifically to a DNA sequence that is within 250 bp of a CpG diresidue in a mammalian cell, fused to
a catalytic domain of Tet2 comprising amino acids corresponding to amino acids 1418-2136 of Tet1 (SEQ ID NO:2), that catalyzes hydroxylation of methylated cytosines in DNA.

24. The fusion protein of claim 23, further comprising a linker between the DNA binding domain and the catalytic domain.

25. The fusion protein of claim 23, comprising a plurality of catalytic domains.

26. The fusion protein of claim 23, wherein the DNA-binding domain comprises a six-finger zinc finger protein.

27. The fusion protein of claim 23, wherein the catalytic domain of Tet2 consists of amino acids corresponding to amino acids 1418-2136 of Tet1 (SEQ ID NO:4).

28. The fusion protein of claim 23, wherein the catalytic domain of Tet2 comprises amino acids corresponding to amino acids 1580-2052 of SEQ ID NO:2, and excluding amino acids corresponding to 584-624 of SEQ ID NO:2.

29. The fusion protein of claim 23, wherein the catalytic domain of Tet2 consists of amino acids corresponding to amino acids 1580-2052 of SEQ ID NO:2, and excluding amino acids corresponding to 584-624 of SEQ ID NO:2

30. A method of reducing methylation of a CpG diresidue in a mammalian cell, the method comprising contacting the cell with a fusion protein of claim 1, wherein the fusion protein binds to a DNA sequence that is within about 250 bp of the CpG diresidue, and wherein the fusion protein reduces methylation of the CpG diresidue.

31. The method of claim 30, wherein the cell is a human cell.

32. The method of claim 30, wherein the DNA sequence is a gene selected from the group consisting of RASSF1A, MGMT, BRCA1, p16, CDH1, TIMP-3, p15, p73, DAPK, APC, and RELN.

33. The method of claim 30, wherein the DNA sequence is within about 100 bp of the CpG diresidue.

34. The method of claim 30, wherein the DNA sequence is within about 30 bp of the CpG diresidue.
